(19) United States
(12) Patent Application Publication
(10) Pub. No.: US 2005/0221306 A1
(43) Pub. Date: Oct. 6, 2005

(54) Detection of predisposition to osteoporosis

(76) Inventors: Scott Geoffrey Wilson, Ocean Reef (AU); Eva Gunvor Kristina Sallstrom, Uppsala (SE); Peter Wayne Reed, Rotorua (NZ); Aruna Bansal, Hertfordshire (GB)

Correspondence Address: BIOTECHNOLOGY LAW GROUP, c/o PORTFOLIO IP, P.O. BOX 52050, MINNEAPOLIS, MN, 55402, US

(21) Appl. No.: 10/495,594

(22) PCT Filed: Oct. 24, 2002
(86) PCT No.: PCT/GB02/04809

§ 371 Date: Apr. 14, 2005

(30) Foreign Application Priority Data
Nov. 3, 2001 (GB) 0126436.5

(51) Int. Cl. C12Q 1/68; C12M 1/34; C07H 21/04

(52) U.S. Cl. 435/6; 435/287.2; 536/24.3

(57) ABSTRACT

The invention provides novel reagents, kits, and methods for diagnosis of predisposition to low spine bone mineral density, low total hip bone mineral density, low femoral neck bone mineral density, or osteoporosis, based on analysis of polymorphic variants of the nucleic acid set forth in SEQ ID NO:1.

The present invention relates to oligonucleotides, kits, microarrays, and methods for detection of bone disease, in particular, osteopenia and osteoporosis.

### BACKGROUND OF THE INVENTION

Osteoporosis is a disease characterized by low bone mass and microarchitectural deterioration of bone tissue, leading to enhanced bone fragility and consequently increased bone fracture. The cost of osteoporotic fracture in the US alone is estimated at $13.8 billion per annum. Fracture is the clinical endpoint in osteoporosis, but bone mineral density (BMD) is commonly used as a surrogate for determining risk of fracture. BMD is an estimate of the mineral mass, corrected for the area (anteroposterior projection) of the bone under study. BMD is the strongest predictor of osteoporotic fracture known, and this measurement is made using Dual X-Ray Absorptiometry (DEXA). Data derived from DEXA scans of the lumbar spine (L1-L4) and hip (total hip and femoral neck) can be used in studies of osteoporosis.

In women, peak bone mass is reached between age 20-30, after which there is a period of consolidation and gradual loss. However, there is a rapid decline in BMD of approximately 3% per year for approximately 5 years after menopause. Consequently, estrogen is strongly implicated as a crucial element in the maintenance of bone mass. This is supported by the role of hormone replacement therapy in the prevention and treatment of osteoporosis. A role for estrogen has also been implicated in the growth and maintenance of bone in men.

Twin and family resemblance studies show that osteoporosis has a substantial genetic component. Data shows that bone mineral density (BMD) has a heritability in the range of 0.66 to 0.82. Other data on Colles' (wrist) fracture in families, also supports the genetic basis of osteoporotic fracture. This data shows that the relative risk of Colles' fracture, if a woman's mother or sisters have had a Colles' fracture, is 1.3 and 1.9 respectively. On the basis of this, and other published data, osteoporosis is regarded as a complex multifactorial disease with multiple gene and environmental factors contributing to disease susceptibility. Lifestyle changes such as moderate exercise; ensuring sufficient calcium intake, limiting alcohol intake; and cessation of smoking, are indicated for individuals with osteopenia and osteoporotic bone disease. Osteopenia and osteoporosis can be treated using lifestyle changes, hormone replacement therapy (HRT: estrogen), selective estrogen receptor modulators (SERMs), antiresorptive agents (bisphosphonates), calcitonin and sodium fluoride. The optimal treatment for osteoporosis has not been determined.

A number of genes have been identified which are associated with predisposition to osteoporosis. For example, U.S. Pat. No. 5,922,542 discloses an association between risk of developing osteoporosis and certain polymorphisms in the gene encoding collagen 1α1, also referred to as COL1A1, and a kit based on this association has been commercialized in Europe.

U.S. Pat. No. 5,998,137 discloses methods of diagnosing a number of diseases, including osteoporosis, by detecting a polymorphism in the promoter of the transforming growth factor β (TGF-β) gene. WO 00/23618 discloses a method of detecting predisposition to osteoporosis on the basis of a polymorphism in intron 5 of the TGF-β gene. JP2000270897 also discloses a method for detection of danger of osteoporosis based on a polymorphism in the TGF-β gene.

U.S. Pat. No. 5,698,399 discloses methods of diagnosing predisposition to osteoporosis by detecting certain polymorphic variants in the gene encoding interleukin-1 receptor antagonist.

U.S. Pat. No. 6,066,450 discloses methods for detecting predisposition to osteoporosis by identifying certain polymorphic variants in the interleukin-6 gene.

EP 1054066 discloses a method for determining sensitivity to an osteoporosis medication based on analysis of certain polymorphisms in the genes encoding vitamin D receptor, the estrogen receptor, and apolipoprotein E.

WO 01/09383 suggests that polymorphisms in the human gene encoding the melatonin-related receptor, a G-protein coupled receptor of unknown function, may be involved in bone-related disorders, including osteoporosis.

WO 01/23559 suggests that mutations in the regulatory region of the human gene encoding osteoclast differentiation factor may be used to detect or predict susceptibility to bone diseases, including osteoporosis.

WO 01/20031 discloses that certain single nucleotide polymorphisms (SNPs) in the human klotho gene are associated with forearm and spine bone mineral density and thus may be used to diagnose predisposition to osteoporosis.

Because osteoporosis is likely to be the result of genetic variations in multiple genes, additional genes and polymorphisms which may be associated with this condition are the subject of ongoing research. A need remains for identification of such genes, to enhance physicians' ability to detect osteopenia and predisposition to osteoporosis in individuals who may have the condition, but do not exhibit symptoms. Thus lifestyle changes or therapy can be initiated early in the progression of disease.

### SUMMARY OF THE INVENTION

On the basis of genetic analysis of dizygotic twins and sib pairs, the present inventors have discovered that certain polymorphisms in the nucleic acid set forth in SEQ ID NO:1 are associated with variation in bone mineral density (BMD). Specifically, the presence of an A nucleotide at position 245 of SEQ ID NO:1 is associated with low spine BMD, low total hip BMD, and low femoral neck BMD, and the presence of a C nucleotide at position 245 of SEQ ID NO:1 is associated with higher spine, total hip, and femoral neck BMD. In addition, the presence of a G nucleotide at position 1470 of SEQ ID NO:1 is associated with low total hip and femoral neck BMD, and the presence of an A nucleotide at position 1470 of SEQ ID NO:1 is associated with higher total hip and femoral neck BMD. Moreover, the presence of a haplotype represented by an A nucleotide at position 245 of SEQ ID NO:1 and a G nucleotide at position 1470 of SEQ ID NO:1 is associated with low spine BMD, low total hip BMD, and low femoral neck BMD. These associations may be used as the basis of reagents, kits, and methods for detection of predisposition to osteoporosis.

The nucleic acid of SEQ ID NO:1 is a draft human genomic sequence of nucleotides 58007412 to 58016140 of chromosome 3 (Human Genome Project Working Draft, University of California, Santa Cruz, April 2001 Freeze), which comprises a cDNA (GenBank Accession No. XM_{—}003213) corresponding to a gene of unknown function currently known as E2IG3. The complete genomic sequence of E2IG3 is available in a chromosome 3 working draft sequence with GenBank Accession No. NT_{—}005986 [gi:15297785]. E2IG3 is upregulated by 17β estradiol (estrogen). The protein encoded by E2IG3 is believed to belong to a subfamily of large GTP-binding proteins. The E2IG3 cDNA is also disclosed as SEQ BD NO:247 of WO 00/55350 and as SEQ ID NO: 6137 of WO 00/58473.

In one embodiment, the invention provides a sequence determination oligonucleotide complementary to a polymorphic region within a nucleic acid having a sequence as set forth in SEQ ID NO:1, wherein the region corresponds to a polymorphic site selected from the group consisting of position 245 of SEQ ID NO:1 and position 1470 of SEQ ID NO:1.

In another-embodiment, the invention provides a microarray comprising at least one oligonucleotide complementary to a polymorphic region in the nucleic acid set forth in SEQ ID NO:1, wherein the region corresponds to a polymorphic site selected from the group consisting of position 245 of SEQ ID NO:1 and position 1470 of SEQ ID NO:1.

In another embodiment, the invention provides the oligonucleotide primer pairs useful for amplification of a polymorphic region in the nucleic acid of SEQ ID NO:1 from a biological sample, wherein the region corresponds to a polymorphic site selected from the group consisting of position 245 of SEQ ID NO:1 and position 1470 of SEQ ID NO:1.

In another embodiment, the invention provides a kit comprising at least one oligonucleotide primer pair complementary to a polymorphic region of the nucleic acid of SEQ ID NO:1, wherein the region corresponds to a polymorphic site selected from the group consisting of position 245 of SEQ ID NO:1 and position 1470 of SEQ ID NO:1.

The invention is also embodied in a method of diagnosing predisposition to low spine bone mineral density, low total hip bone mineral density, low femoral neck bone mineral density, or osteoporosis in a human, said method comprising the steps of obtaining a nucleic acid sample from the human; detecting the presence or absence of at least one allelic variant of a polymorphic region in a nucleic acid having a sequence as set forth in SEQ ID NO:1 in the sample, wherein the polymorphic region corresponds to the polymorphic site at position 245 of SEQ ID NO:1.

The invention is also embodied in a method of diagnosing predisposition to low total hip bone mineral density, low femoral neck bone mineral density, or osteoporosis in a human, said method comprising the steps of obtaining a nucleic acid sample from the human; and detecting the presence or absence of at least one allelic variant of a polymorphic region in a nucleic acid having a sequence as set forth in SEQ ID NO:1 in the sample, wherein the polymorphic region corresponds to the polymorphic site at position 1470 of SEQ ID NO:1.

In a further embodiment, the invention provides a method of diagnosing predisposition to low spine bone mineral density, low total hip bone mineral density, low femoral neck neck bone mineral density, or osteoporosis in a human comprising the steps of obtaining a nucleic acid sample from the human; and detecting the presence or absence of a haplotype of the nucleic acid having a sequence as set forth in SEQ ID NO:1, said haplotype being characterized by: an A nucleotide at position 245 of SEQ ID NO:1 and a G nucleotide at position 1470 of SEQ ID NO:1.

### BRIEF DESCRIPTION OF THE DRAWINGS

FIG. 1 sets forth the nucleic acid of SEQ ID NO:1 with the polymorphic sites at positions 245 and 1470 in bold capital type. The transcription start site of E2IG3 is indicated in italics, introns are depicted in lower case type, and exons are depicted in bold lower case type. Exons are as described in GenBank Accession No. XM_{—}003213.

FIG. 2 sets forth the sequences of certain oligonucleotides of the invention, which are correlated with the polymorphic site the oligonucleotides are designed to detect. Polymorphic sites in these oligonucleotides are indicated in bold capital type.

FIG. 3 sets forth the sequences of certain oligonucleotide primer pairs designed to amplify polymorphic regions of the nucleic acid of SEQ ID NO:1.

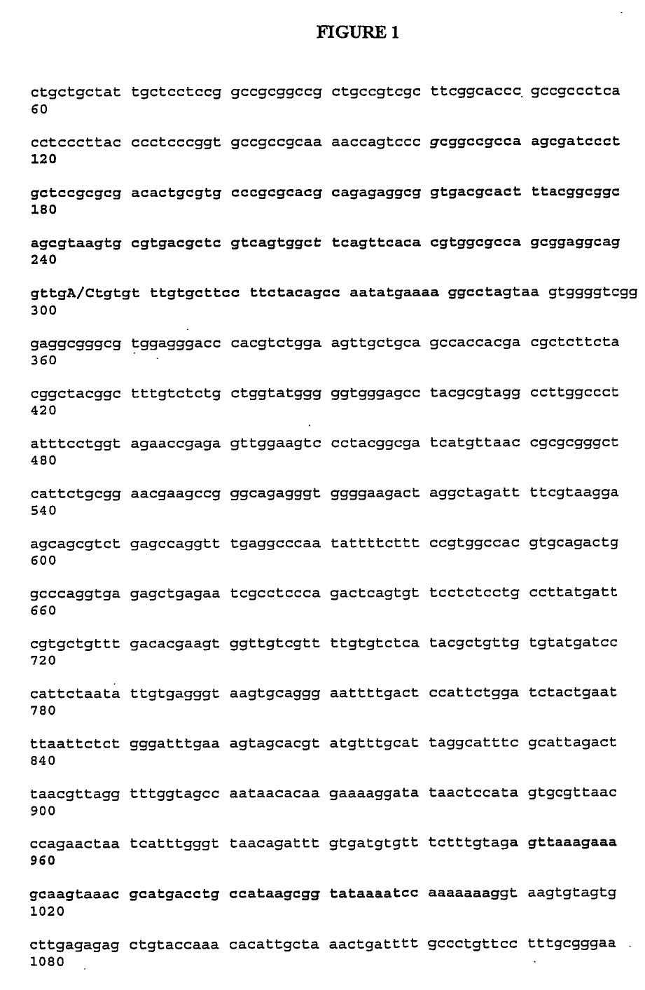

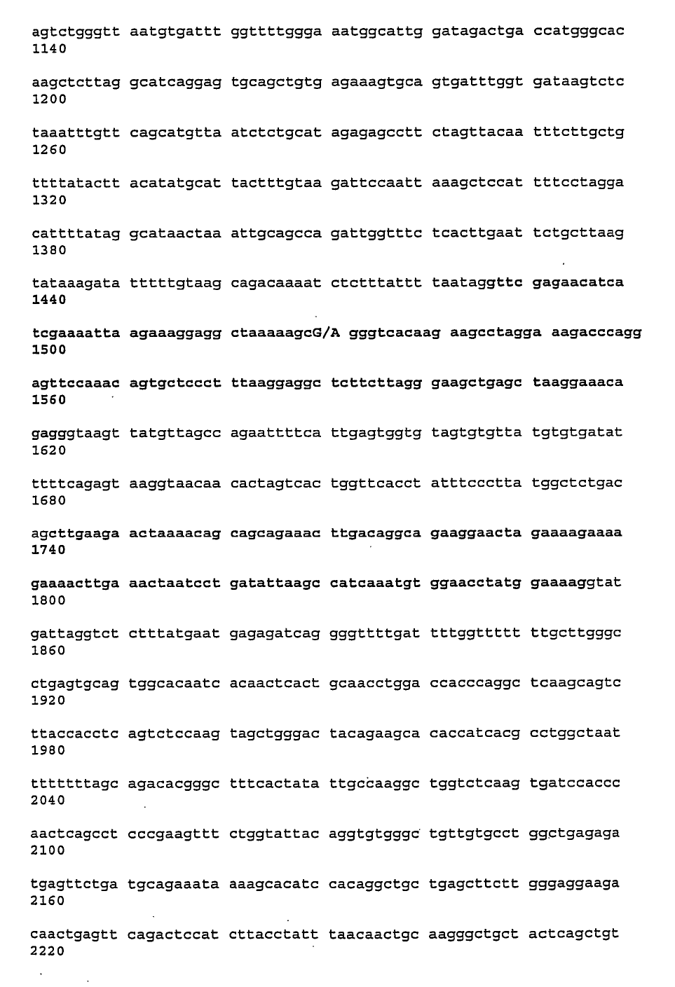

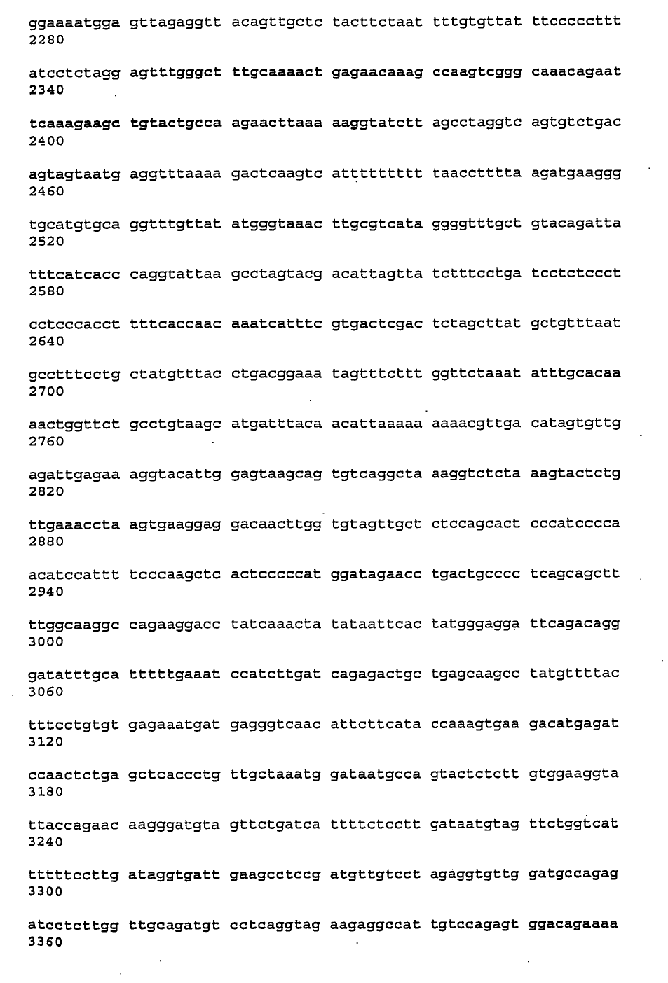

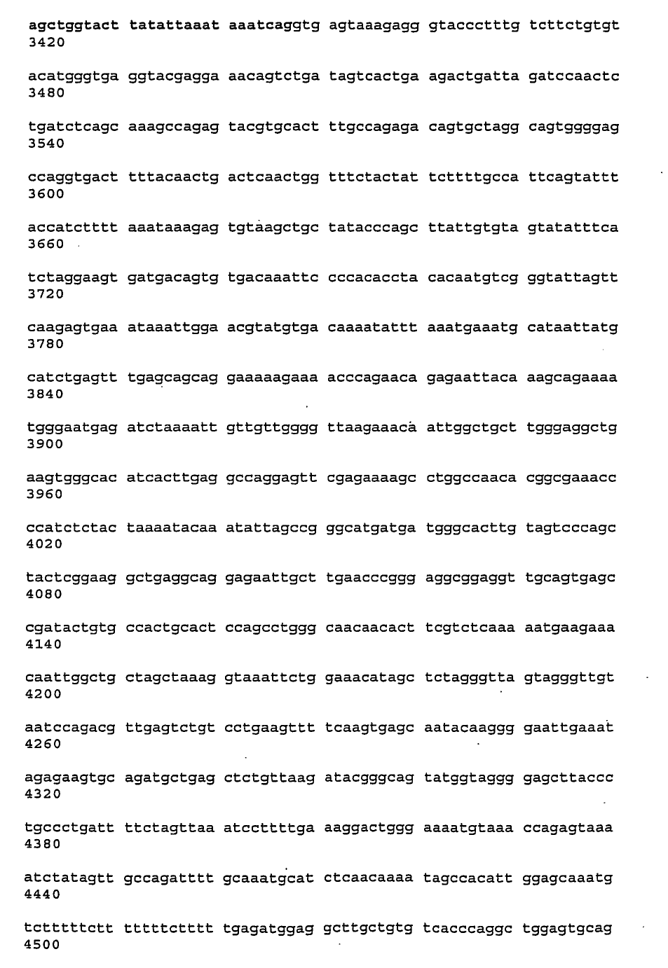

### DETAILED DESCRIPTION OF THE INVENTION

The U.S. patents and publications referenced herein are hereby incorporated by reference. Examples 1 and 2 below demonstrate associations between certain polymorphic regions in SEQ ID NO:1 and measurements of bone mineral density.

For the purposes of the invention, certain terms are defined as follows.

“Oligonucleotide” means a nucleic acid molecule preferably comprising from about 8 to about 50 covalently linked nucleotides. More preferably, an oligonucleotide of the invention comprises from about 8 to about 35 nucleotides. Most preferably, an oligonucleotide of the invention comprises from about 10 to about 25 nucleotides. In accordance with the invention, the nucleotides within an oligonucleotide may be analogs or derivatives of naturally occurring nucleotides, so long as oligonucleotides containing such analogs or derivatives retain the ability to hybridize specifically within the polymorphic region containing the targeted polymorphism. Analogs and derivatives of naturally occurring oligonucleotides within the scope of the present invention are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and the like. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372, and the like. The term “oligonucleotides” as defined herein includes compounds which comprise the specific oligonucleotides disclosed herein, covalently linked to a second moiety. The second moiety may be an additional nucleotide sequence, for example, a tail sequence such as a polyadenosine tail or an adaptor sequence, for example, the phage M13 universal tail sequence, and the like. Alternatively, the second moiety may be a non-nucleotidic moiety, for example, a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the specific oligonucleotide, so long as the oligonucleotide retains its ability to hybridize to the polymorphic regions described herein.

A polymorphic region as defined herein is a portion of a genetic locus that is characterized by at least one polymorphic site. A genetic locus is a location on a chromosome which is associated with a gene, a physical feature, or a phenotypic trait. A polymorphic site is a position within a genetic locus at which at least two alternative sequences have been observed in a population. A polymorphic region as defined herein is said to “correspond to” a polymorphic site, that is, the region may be adjacent to the polymorphic site on the 5′ side of the site or on the 3′ side of the site, or alternatively may contain the polymorphic site. A polymorphic region includes both the sense and antisense strands of the nucleic acid comprising the polymorphic site, and may have a length of from about 100 to about 5000 base pairs. For example; a polymorphic region may be all or a portion of a regulatory region such as a promoter, 5′ UTR, 3′ UTR, an intron, an exon, or the like. A polymorphic or allelic variant is a genomic DNA, cDNA, mRNA or polypeptide having a nucleotide or amino acid sequence that comprises a polymorphism. A polymorphism is a sequence variation observed at a polymorphic site, including nucleotide substitutions (single nucleotide polymorphisms or SNPs), insertions, deletions, and microsatellites. Polymorphisms may or may not result in detectable differences in gene expression, protein structure, or protein function. Preferably, a polymorphic region of the present invention has a length of about 1000 base pairs. More preferably, a polymorphic region of the invention has a length of about 500 base pairs. Most preferably, a polymorphic region of the invention has a length of about 200 base pairs.

A haplotype as defined herein is a representation of the combination of polymorphic variants in a defined region within a genetic locus on one of the chromosomes in a chromosome pair. A genotype as used herein is a representation of the polymorphic variants present at a polymorphic site.

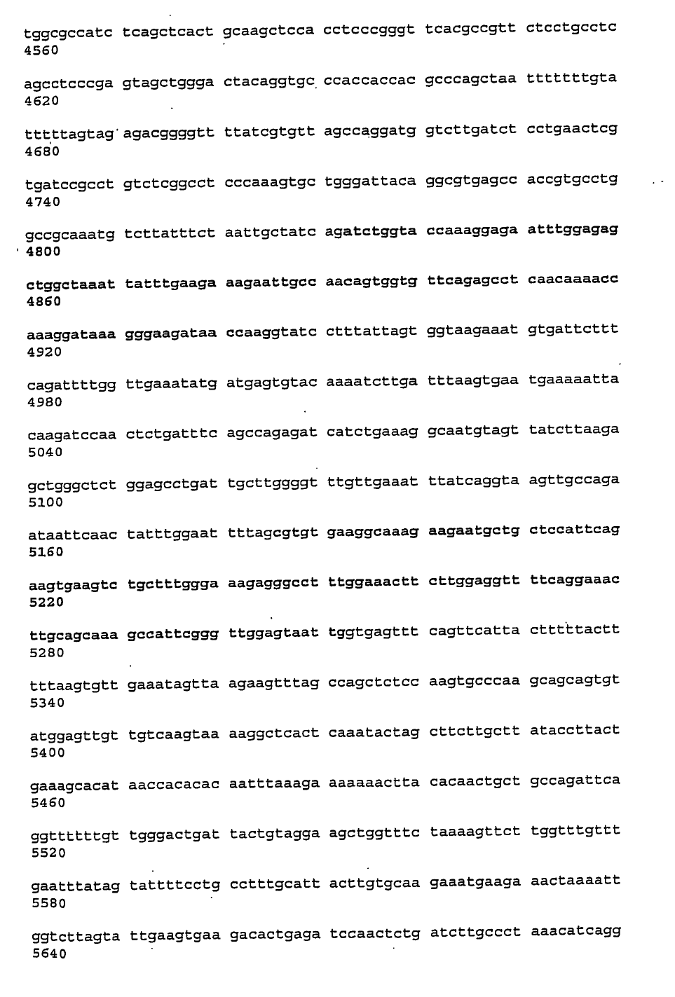

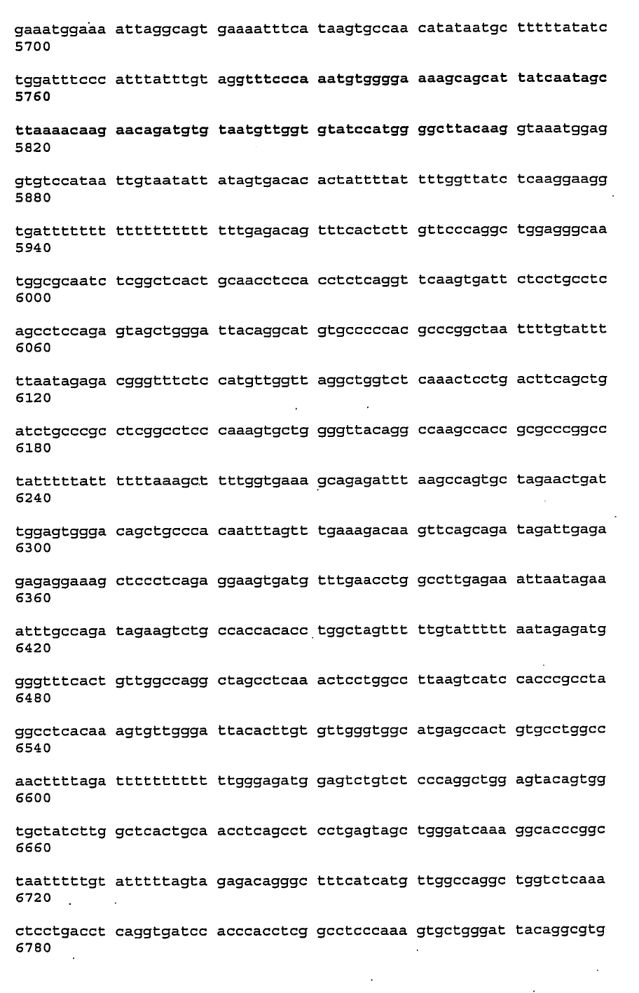

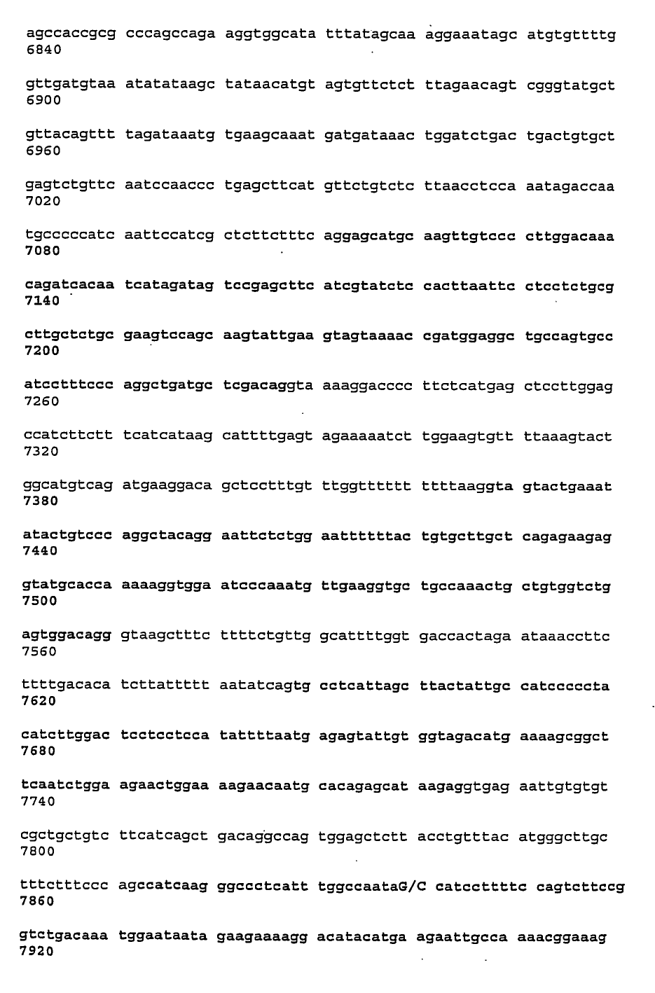

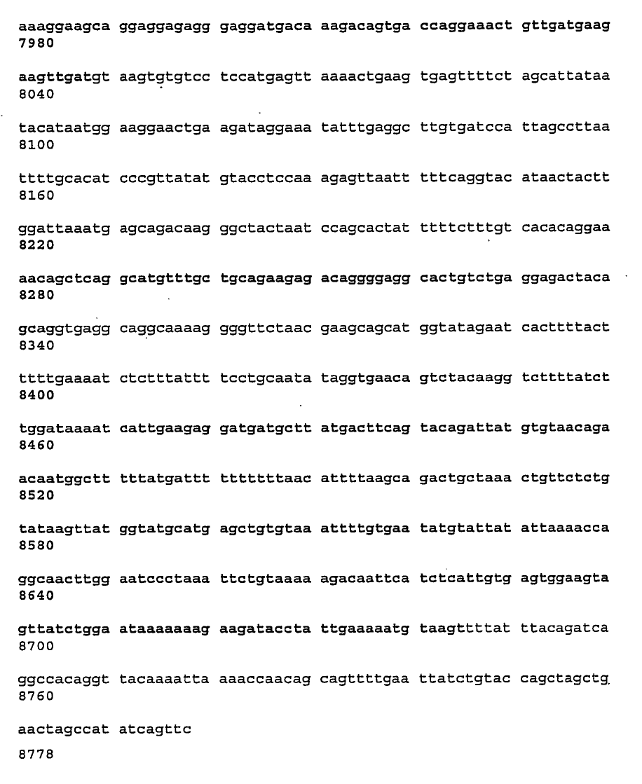

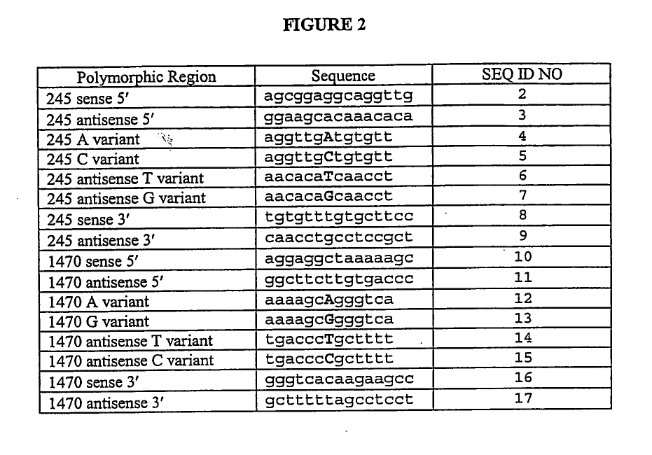

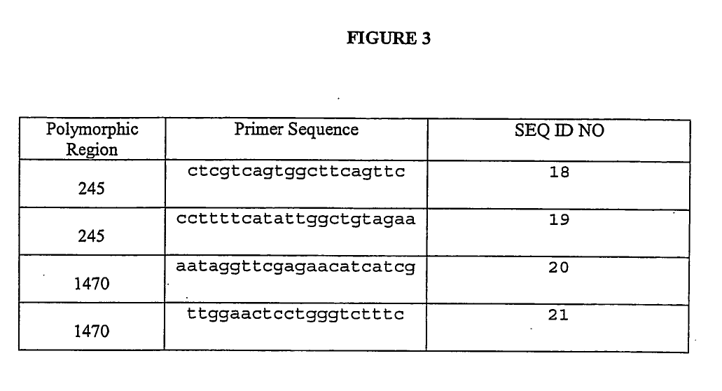

A polymorphic region of the present invention comprises a portion of SEQ ID NO:1 corresponding to at least one of the polymorphic sites identified above. That is, a polymorphic region of the invention may include a nucleotide sequence surrounding and/or including any of the polymorphic sites at positions 245 and 1470 of SEQ ID NO:1. Polymorphic regions in the antisense nucleic acid complementary to SEQ ID NO:1 are also encompassed in the present invention, wherein the region includes a nucleotide sequence surrounding and/or including any of the antisense positions corresponding to positions 245 and 1470 of SEQ ID NO:1. FIG. 2 sets forth exemplary oligonucleotides within the scope of this embodiment. For example, a polymorphic region corresponding to the polymorphic site at position 245 of SEQ ID NO:1 may comprise a sequence as set forth in any of SEQ ID NO:2; SEQ ID NO:3; SEQ ID NO:4; SEQ ID NO:5; SEQ ID NO:6; SEQ ID NO:7; SEQ ID NO:8; or SEQ ID NO:9. A polymorphic region corresponding to the polymorphic site at position 1470 of SEQ ID NO:1 may comprise a sequence as set forth in any of SEQ ID NO:10; SEQ ID NO:11; SEQ ID NO:12; SEQ ID NO:13; SEQ ID NO:14; SEQ ID NO:15; SEQ ID NO:16; or SEQ ID NO:17.

In certain embodiments of the invention, oligonucleotides are used as probes for the polymorphic regions in the nucleic acid having the sequence set forth in SEQ ID NO:1. These oligonucleotides may also be termed “sequence determination oligonucleotides” within the scope of the invention, and may be used to determine the presence or absence of a particular nucleotide at a particular polymorphic site within the nucleic acid of SEQ ID NO:1. Specific oligonucleotides of the invention include any oligonucleotide complementary to any of the polymorphic regions described above.

Those of ordinary skill will recognize that oligonucleotides complementary to the polymorphic regions described herein must be capable of hybridizing to the polymorphic regions under conditions of stringency such as those employed in primer extension-based sequence determination methods, restriction site analysis, nucleic acid amplification methods, ligase-based sequencing methods, methods based on enzymatic detection of mismatches, microarray-based sequence determination methods, and the like. The oligonucleotides of the invention may be synthesized using known methods and machines, such as the ABI™3900 High Throughput DNA Synthesizer and the EXPEDITE™ 8909 Nucleic Acid Synthesizer, both of which are available from Applied Biosystems (Foster City, Calif.).

The oligonucleotides of the invention may be used, without limitation, as in situ hybridization probes or as components of diagnostic assays. Numerous oligonucleotide-based diagnostic assays are known. For example, primer extension-based nucleic acid sequence detection methods are disclosed in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; WO 01/20039; and the like. Primer extension-based nucleic acid sequence detection methods using mass spectrometry are described in U.S. Pat. Nos. 5,547,835; 5,605,798; 5,691,141; 5,849,542; 5,869,242; 5,928,906; 6,043,031; 6,194,144, and the like. The oligonucleotides of the invention are also suitable for use in ligase-based sequence determination methods such as those disclosed in U.S. Pat. Nos. 5,679,524 and 5,952,174, WO 01/27326, and the like. The oligonucleotides of the invention may be used as probes in sequence determination methods based on mismatches, such as the methods described in U.S. Pat. Nos. 5,851,770; 5,958,692; 6,110,684; 6,183,958; and the like. In addition, the oligonucleotides of the invention may be used in hybridization-based diagnostic assays such as those described in U.S. Pat. Nos. 5,891,625; 6,013,499; and the like.

The oligonucleotides of the invention may also be used as components of a diagnostic microarray. Methods of making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; WO 01/29259; and the like. Preferably, the microarray of the invention comprises at least one oligonucleotide complementary to a polymorphic region of SEQ ID NO:1, wherein the region corresponds to a polymorphic site selected from the group consisting of position 245 of SEQ ID NO:1 and position 1470 of SEQ ID NO:1. More preferably, the microarray of the invention comprises an oligonucleotide complementary to a polymorphic region corresponding to position 245 of SEQ ID NO:1 and an oligonucleotide complementary to a polymorphic region corresponding to position 1470 of SEQ ID NO:1. In a specific embodiment, the oligonucleotides of the microarray of the invention are complementary to any or all of the polymorphic regions selected from the group consisting of SEQ ID NO:2; SEQ ID NO:3; SEQ ID NO:4; SEQ ID NO:5; SEQ ID NO:6; SEQ ID NO:7; SEQ D NO:8; and SEQ ID NO:9 (corresponding to the polymorphic site at position 245 of SEQ ID NO:1); and the polymorphic regions selected from the group consisting of SEQ ID NO:10; SEQ ID NO:11; SEQ ID NO:12; SEQ ID NO:13; SEQ ID NO:14; SEQ ID NO:15; SEQ ID NO:16; and SEQ ID NO:17 (corresponding to the polymorphic site at position 1470 of SEQ ID NO:1).

The invention is also embodied in oligonucleotide primer pairs suitable for use in the polymerase chain reaction (PCR) or in other nucleic acid amplification methods. Each oligonucleotide primer pair of the invention is complementary to a polymorphic region of the nucleic acid of SEQ ID NO:1. Thus an oligonucleotide primer pair of the invention is complementary to a polymorphic region characteristic of at least one of the polymorphic sites at positions 245 and 1470 of SEQ ID NO:1. Those of ordinary skill will be able to design suitable oligonucleotide primer pairs using knowledge readily available in the art, in combination with the teachings herein. Specific oligonucleotide primer pairs of this embodiment include the oligonucleotide primer pairs set forth in SEQ ID NO:18 and SEQ ID NO:19, which are suitable for amplifying the polymorphic region corresponding to the polymorphic site at position 245 of SEQ ID NO:1; and the oligonucleotide primer pairs set forth in SEQ ID NO:20 and SEQ ID NO:21, which are suitable for amplifying the polymorphic region corresponding to the polymorphic site at position 1470 of SEQ ID NO:1. Those of skill will recognize that other oligonucleotide primer pairs suitable for amplifying the polymorphic regions of the nucleic acid of SEQ ID NO:1 can be designed without undue experimentation. In particular, oligonucleotide primer pairs suitable for amplification of larger portions of SEQ ID NO:1 Would be preferred for haplotype analysis.

Each of the PCR primer pairs of the invention may be used in any PCR method. For example, a PCR primer pair of the invention may be used in the methods disclosed in U.S. Pat. Nos. 4,683,195; 4,683,202, 4,965,188; 5,656,493; 5,998,143; 6,140,054; WO 01/27327; WO 01/27329; and the like. The PCR pairs of the invention may also be used in any of the commercially available machines that perform PCR, such as any of the GENEAMP® Systems available from Applied Biosystems.

The invention is also embodied in a kit comprising at least one oligonucleotide primer pair of the invention. Preferably, the kit of the invention comprises at least two oligonucleotide primer pair, wherein each primer pair is complementary to a different polymorphic region of the nucleic acid of SEQ ID NO:1. More preferably, the kit of the invention comprises at least one oligonucleotide primer pair suitable for amplification of polymorphic regions corresponding to positions 245 or 1470 of SEQ ID NO:1. This embodiment may optionally further comprise a sequence determination oligonucleotide for detecting a polymorphic variant at any or all of the polymorphic sites corresponding to positions 245 and 1470 of SEQ ID NO:1. The kit of the invention may also comprise a polymerizing agent, for example, a thermostable nucleic acid polymerase such as those disclosed in U.S. Pat. Nos. 4,889,818; 6,077,664, and the like. The kit of the invention may also comprise chain elongating nucleotides, such as dATP, dTTP, dGTP, dCTP, and dITP, including analogs of dATP, dTTP, dGTP, dCTP and dITP, so long as such analogs are substrates for a thermostable nucleic acid polymerase and can be incorporated into a growing nucleic acid chain. The kit of the invention may also include chain terminating nucleotides such as ddATP, ddTTP, ddGTP, ddCTP, and the like. In a preferred embodiment, the kit of the invention comprises at least one oligonucleotide primer pair, a polymerizing agent, chain elongating nucleotides, at least one sequence determination oligonucleotide and at least one chain terminating nucleotide. The kit of the invention may optionally include buffers, vials, microtiter plates, and instructions for use.

Methods of diagnosing predisposition to low spine bone mineral density, low total hip bone mineral density, low femoral neck bone mineral density, or osteoporosis in a human are also encompassed by the present invention. In the methods of the invention, the presence or absence of at least one polymorphic variant of the nucleic acid of SEQ ID NO:1 is detected to determine or diagnose such a predisposition. Specifically, in a first step, a nucleic acid is isolated from biological sample obtained from the human. Any nucleic-acid containing biological sample from the human is an appropriate source of nucleic acid for use in the methods of the invention. For example, nucleic acid can be isolated from blood, saliva, sputum, urine, cell scrapings, biopsy tissue, and the like. In a second step, the nucleic acid is assayed for the presence or absence of at least one allelic variant of any or all of the polymorphic regions of the nucleic acid of SEQ ID NO:1 described above. Preferably, the polymorphic regions on both chromosomes in the chromosome pair of the human are assayed in the method of the invention, so that the zygosity of the individual for the particular polymorphic variant may be determined.

Any method may be used to assay the nucleic acid, that is, to determine the sequence of the polymorphic region, in this step of the invention. For example, any of the primer extension-based methods, ligase-based sequence determination methods, mismatch-based sequence determination methods, or microarray-based sequence determination methods described above may be used, in accordance with the present invention. Alternatively, such methods as restriction fragment length polymorphism (RFLP) detection, single strand conformation polymorphism detection (SSCP), PCR-based assays such as the TAQMAN® PCR System (Applied Biosystems) may be used.

In accordance with one method of the invention, predisposition to low spine bone mineral density, low total hip bone mineral density, low femoral neck bone mineral density, or osteoporosis is diagnosed by determining the identity of the nucleotide at position 245 of SEQ ID NO:1. In this method, an A nucleotide at position 245 of SEQ ID NO:1, or a T nucleotide at the corresponding position of the antisense complement of SEQ ID NO:1, is indicative of greater risk of developing low spine bone mineral density, low total hip bone mineral density, low femoral neck bone mineral density, or osteoporosis.

Conversely, the presence of a C nucleotide at position 245 of SEQ ID NO:1, or of a G nucleotide at the corresponding position of the antisense complement of SEQ ID NO:1, is indicative of a lower risk of developing low spine bone mineral density, low total hip bone mineral density, low femoral neck bone mineral density, or osteoporosis. In a further step, the zygosity of the individual may be determined, wherein a homozygous AA genotype at position 245 of SEQ ID NO:1 or TT genotype at the corresponding position of the antisense complement of SEQ ID NO:1, indicates greatest risk for developing low spine bone mineral density, low total hip bone mineral density, low femoral neck bone mineral density, or osteoporosis. A person whose genotype is homozygous CC at position 245 of SEQ ID NO:1 or GG at the corresponding position of the antisense complement of SEQ ID NO:1 is at least risk for developing low spine bone mineral density, low total hip bone mineral density, low femoral neck bone mineral density, or osteoporosis. An individual whose genotype is heterozygous AC at position 245 of SEQ ID NO:1 or TG at the corresponding position of the antisense complement of SEQ ID NO:1 is at intermediate risk for developing low spine bone mineral density, low total hip bone mineral density, low femoral neck bone mineral density, or osteoporosis. Alternatively, predisposition to low total hip bone mineral density, low femoral neck bone mineral density, or osteoporosis is diagnosed by determining the identity of the nucleotide at position 1470 of SEQ ID NO:1. In this embodiment, predisposition to low total hip bone mineral density, low femoral neck bone mineral density, or osteoporosis is diagnosed by determining the identity of the nucleotide at position 1470 of SEQ ID NO:1. In this method, a G nucleotide at position 1470 of SEQ ID NO:1, or a C nucleotide at the corresponding position of the antisense complement of SEQ ID NO:1, is indicative of greater risk of developing low total hip bone mineral density, low femoral neck bone mineral density, or osteoporosis. Conversely, the presence of an A nucleotide at position 1470 of SEQ ID NO:1, or of a T nucleotide at the corresponding position of the antisense complement of SEQ ID NO:1, is indicative of a lower risk of developing low total hip bone mineral density, low femoral neck bone mineral density, or osteoporosis. In a further step, the zygosity of the individual may be determined, wherein a homozygous GG genotype at position 1470 of SEQ D NO: 1 or CC genotype at the corresponding position of the antisense complement of SEQ ID NO:1, indicates greatest risk for developing low total hip bone mineral density, low femoral neck bone mineral density, or osteoporosis. A person whose genotype is homozygous AA at position 1470 of SEQ ID NO:1 or TT at the corresponding position of the antisense complement of SEQ ID NO:1 is at least risk for developing low total hip bone mineral density, low femoral neck bone mineral density, or osteoporosis. An individual whose genotype is heterozygous GA at position 1470 of SEQ ID NO:1 or CT at the corresponding position of the antisense complement of SEQ ID NO:1 is at intermediate risk for developing low total hip bone mineral density, low femoral neck bone mineral density, or osteoporosis. In another method of the invention, risk of low spine bone mineral density, low total hip bone mineral density, low femoral neck bone mineral density, or osteoporosis is assessed by determining the haplotype of the individual for both polymorphic positions within SEQ ID NO:1. For example, individuals who possess the SEQ ID NO:1 haplotype characterized by an A nucleotide at position 245 of SEQ ID NO:1 and a G nucleotide at position 1470 of SEQ ID NO:1 are at higher risk of development of low spine bone mineral density, low total hip bone mineral density, low femoral neck bone mineral density, or osteoporosis. This haplotype may alternatively be detected on the antisense complement of SEQ ID NO:1 as a T nucleotide at the antisense position corresponding to position 245 of SEQ ID NO:1 and a C nucleotide at the antisense position corresponding to position 1470 of SEQ ID NO:1. Individuals who are homozygous for allelic variants comprising this haplotype are at particularly high risk of developing low spine bone mineral density, low total hip bone mineral density, low femoral neck bone mineral density, or osteoporosis.

The examples set forth below are provided as illustration and are not intended to limit the scope and spirit of the invention as specifically embodied therein.

### EXAMPLE 1

### Clinical Samples

A study design based on extreme discordant and concordant sib pairs (EDACS) was chosen for analysis. All available female sibs of families containing EDACS were included in the sample. EDACS were defined on the criteria of BMD Z score. In each family, probands required a BMD Z for total spine (L1-4), total hip or femoral neck of Z≦−1.5. At least one additional sib in the family was required to have either BMD for total spine (L1-4), total hip or femoral neck equal to Z≦−1.0 or Z≦1.0. Z scores were derived from individual BMD scan data (Hologic) of the subjects and transformed using the published Hologic reference range for spine BMD (Favus, 1999 in Primer on the metabolic bone diseases and disorders of mineral metabolism, Ed. Favus, M J, 4^{th} Edition, Lippincott Williams and Wilkins, Philadelphia, USA. 1999:483-484 or NHANESIII (Looker et al. (1995) J Bone Mineral Res. 10, 796-802) for hip and femoral neck. Any BMD data collected using Lunar technology was transformed to the Hologic BMD equivalent using sBMD (Genant et al. (1994) J Bone Mineral Res. 9:1503-14; Steiger et al. (1995) J Bone Mineral Res. 10:1602; Hanson et al. (1997) J Bone Mineral Res. 12:1316).

A BMD Z score <−1.5 or >+1.5 corresponds to the bottom or top 6.7% of the age matched distribution, respectively. A Z score of −1.0 or +1.0 corresponds to being in the bottom or top 15.9% of the age matched distribution and includes both osteoporotic and osteopenic individuals. A total of 1098 samples were selected and plated for analysis as the study sample set.

Whole genome scans were performed on 1401 twin pairs using up to 706 highly polymorphic microsatellite markers (Reed et al. (1994) Nat Genet 7:390-5). Additional whole genome scans were performed on 649 subjects from 283 families containing a proband with low bone mineral density. These later subjects were genome scanned using the Affymetrix HuSNP GENECHIP® technology platform (Wilson et al. (2000) Calcified Tissue International 67:484). Multipoint nonparametric linkage analysis was performed using MAPMAKER/SIBS (Kruglyak & Lander (1995) Amer. J. Human Genetics 57:439-454).

For fine mapping and SNP genotyping a study design based on extreme exclusion criteria for probands and sibs were used and verified by questionnaire if possible. These exclusion criteria were myeloma, osteosarcoma, or malignancy with skeletal involvement, hyperparathyroidism, unstable thyroid disease, long term steroid use (>5 mg/day for more than 6 months and presently on therapy), chronic immobility, rheumatoid arthritis, anorexia nervosa (>1 yr), history of osteomalacia, amenorrhea for >6 months, premature cessation of regular menstruation or surgical oophorectomy +/−/HRT (age <35 yrs), very late menarche >20 years of age, 15>BMI>40, and epilepsy with use of anticonvulsant medication for >1 year. These exclusions were applied conservatively and only subjects who had substantial evidence that they should be excluded were removed, because it was not possible to verify the exact details with the patients or the medical records.

### EXAMPLE 2

### Genotyping

The linkage studies used a proprietary bioinformatics infrastructure and proprietary software packages to record marker positions, store data and generate data files (See WO 00/51053). Output from these systems was then used with the relevant application software to perform the statistical analysis.

MAPMAKER/SIBS (Kruglyak & Lander (1995) Amer. J Human Genetics 57, 439-454) was used to estimate multipoint nonparametric linkage for the fine mapping studies. The computer program QTDT (Abecasis et al. (2000) Amer. J Human Genetics 66, 279-292) was used to test for evidence of association. Three tests were considered: Transmission/disequilibrium test (TDT), population stratification and total association tests. Population stratification can lead to spurious results from total association testing, so the latter result was considered and reported only when there was no evidence of population stratification.

Haplotype analysis was with QPDT (Martin et al. (2000) Amer. J. Human Genetics 67, 146-54), which utilises the EM (Dempster et al. (1977) J Royal Statistical Soc. B39, 1-38) algorithm to assign haplotypes based on likelihood maximization.

A. Microsatellite Fine Mapping

Thirty-five microsatellite markers in a broad interval (50.4 cM-111 cM) on chromosome 3 were chosen for analysis in the study population, with a mean spacing of 2.02 cM between each marker, across the region. Genotyping reactions were generally carried out in microtitre plates (384-well, reaction volume 5 μl), containing 12.5 ng of DNA DNA from study subjects was amplified using PCR and sequence specific oligonucleotide primers labelled with 6-FAM™, HEX™, or NED™ fluorescent dyes. PCR products were analysed by electrophoresis in a polyacrylamide denaturing gel, with an ABI PRISM™ GENESCAN® 400HD ROX labelled size standard in each lane on an ABI model 377 analyzer (Applied Biosystems, Foster City, Calif.). For genotyping, the chosen markers were divided into two groups (panels) so that the analysis of all of the markers could be performed in two electrophoresis runs of each sample. Consequently, there was no overlap of fragment sizes in any one dye for either of the panels. Genotype analysis was performed using ABI PRISM™ GENESCAN® software (version 3.0), and genotyped manually using ABI PRISM™ Genotyper 2.0. Results were input into a proprietary database and binned by marker. The results were quality checked, ensuring consistent inheritance within families. Families that were found to have consistent pedigree problems were excluded from the analysis set.

The ordering of genetic mapping markers (i.e. microsatellite markers) was relatively stable in the region analyzed according to the Unified Data Base for Human Genome Mapping, Weizmann Institute of Science (UDB) and National Center for Biotechnology Information, National Institutes of Health (NCBI) assemblies during the duration of the study. Conversion of genetic to physical positions for strategic microsatellite markers was performed using UDB and NCBI as the reference standards. Comparisons of the identity and positioning of genomic contigs in the region were also made between UDB and NCBI and provided relatively good agreement. A comparison of the positioning of all identified and predicted genes within the region was also made between NCBI (build 22) and Joint Project between European Bioinformatics Institute and the Sanger Centre (ENSEMBL). At its broadest the region encompassed genomic contigs NT_{—}005980.3 to NT_{—}005607.3 (build 22) or NT_{—}005498.4 to NT_{—}005589.4 (build 24). Given the identified mapping inconsistencies between public domain genome assemblies, several additional contigs were also considered. The major focus within this broad region was between NT_{—}006022.3 and NT_{—}005589.3 (NCBI build 22; 43.9 Mb-57.2 Mb).

The microsatellite marker analysis showed linkage of spine BMD Z to chromosome 3p21 with a non parametric Z=4.07 at 68.8 cM (n=1619 pedigrees). Using the −1 LOD approach, the support interval was 62.16 cM-75.62 cM. Total hip gave a peak Z score within the region of 1.17 at 65.1 cM and femoral neck Z=1.02 at 60 cM.

The additional markers increased the information content for linkage, from a mean of 0.4382 to 0.5623, (range of 0.3177-0.6967). In the region of greatest interest, from 50 cM to 100 cM, information content for linkage has mean 0.6007 and range 0.5357-0.6967.

No association was found using multi-allelic test of association for microsatellite markers within the support interval.

B. SNP Genotyping

SNPs analysed in the study were sourced from the public SNP resource, dbSNP, which contains an estimated 1.4 million non-redundant SNPs mapped to the NCBI genome browser (Stoneking, 2001, Nature 409: 821-822).

SNPs were selected at approximately 50 kb intervals across the region analyzed. The following attributes were taken into account when selecting SNPs: amount of available 5′ and 3′ sequence context, absence of repeat masking in surrounding sequence, presence of multiple submissions, identity and reputation of the submitter. Relatively high SNP failure rate has been predicted, so several rounds of SNP selection were planned and undertaken.

Where SNP validation and screening were performed, the SNP assay was run on a sample set composed of 96 samples and replicated in quadruplicate. Results were examined to verify a working PCR reaction and appropriate PCR product, and to establish that some level of polymorphism existed.

Five SNPs in the E2IG3 gene (also known as Q9UJY0) reported by dbSNP were examined. Two of the SNPs (corresponding to position 245 of SEQ ID NO:1, and position 1470 of SEQ D NO: 1) were genotyped in the complete sample set. The SNPs were amplified (GENEAMP® PCR system 9700, Applied Biosystems) using 4.6 ng of genomic DNA in a total volume of 22.3 μl per well. Genotyping was performed using PSQ™ 96 SNP Reagents Kit 5x96 and SNP detection was subsequently performed using the PSQ 96 platform (Pyrosequencing AB, Uppsala, Sweden). In order to detect any genotyping anomalies, Hardy-Weinberg, haplotype analysis and duplicate control genotypings were performed. No deviation in genotyping results could be seen in the duplicates. The frequency of the three different genotypes for each SNP did not differ significantly from expected values in the Hardy-Weinberg test. Both markers and were found to show significant association by TDT with total hip BMD Z (p=0.0009 and p=0.0045 respectively). Data for femoral neck BMD Z also shows significant association with the two markers (p=0.0424 and p=0.0272 respectively). In comparison, spine BMD showed only a weak association to spine BMD with p-values 0.058 and 0.061 respectively, although haplotypes analysis did show association (p=0.0325 for haplotype AG). Analysis with QPDT gave similar results with the lowest p-value for association being with the SNP corresponding to position 245 of SEQ ID NO:1 and total hip BMD, a p-value of 0.0002. The association with spine BMD was 0.016 and 0.076, respectively. Weight is known to explain a substantial proportion of the variance in BMD at weight bearing sites, and particularly at the hip. Consequently, weight adjusted hip BMD was also studied in relation to the E2IG3 association with hip BMD. After adjustment for weight, evidence of association with total hip BMD remains in the analysis (e.g. position 245 of SEQ ID NO:1; p=0.003).

As a result of these findings fragments covering exon 2, 12, 13 and 14 of E2IG3 were sequenced using solid phase sequencing (AUTOLOAD™ Solid Phase Sequencing kit, Amersham Pharmacia Biotech) and gel electrophoresis on ALFEXPRESS™ sequencers (Amersham Pharmacia Biotech) to identify additional SNPs, between the associated SNPs and in the 3′ end of the gene. One additional SNP has been discovered in Exon 13. This is a G to C polymorphism at position 7840 in SEQ ID NO:1. Preliminary data suggests that the frequency of this SNP is below 5%.

While the invention has been described in terms of the specific embodiments set forth above, those of skill will recognize that the essential features of the invention may be varied without undue experimentation and that such variations are within the scope of the appended claims.

1. A microarray comprising at least one oligonucleotide complementary to a polymorphic region within a nucleic acid having a sequence as set forth in SEQ ID NO:1, wherein the region corresponds to a polymorphic site selected from the group consisting of position 245 of SEQ ID NO:1 and position 1470 of SEQ ID NO:1.

2. The microarray of claim 1, comprising an oligonucleotide complementary to a polymorphic region corresponding to position 245 of SEQ ID NO:1 and an oligonucleotide complementary to a polymorphic region corresponding to position 1470 of SEQ ID NO:1.

3. An oligonucleotide complementary to a polymorphic region within a nucleic acid having a sequence as set forth in SEQ ID NO:1, wherein the region corresponds to a polymorphic site selected from the group consisting of position 245 of SEQ ID NO:1 and position 1470 of SEQ ID NO:1.

4. The oligonucleotide of claim 3, wherein the region comprises a sequence selected from the group consisting of SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; and SEQ ID NO:17.

5. A pair of oligonucleotide primers for amplifying a polymorphic region in a nucleic acid having a sequence as set forth in SEQ ID NO:1 from a biological sample, wherein the region corresponds to a polymorphic site selected from the group consisting of position 245 of SEQ ID NO:1 and position 1470 of SEQ ID NO:1

6. The primers of claim 5, having sequences selected from the group consisting of: SEQ ID NO:18 and SEQ ID NO:19; and SEQ ID NO:20 and SEQ ID NO:21.

7. A kit comprising at least one oligonucleotide primer pair complementary to a polymorphic region of a nucleic acid having a sequence as set forth in SEQ ID NO:1, wherein the region corresponds to a polymorphic site selected from the group consisting of position 245 of SEQ ID NO:1 and position 1470 of SEQ ID NO:1.

8. The kit of claim 7, comprising at least two oligonucleotide primer pairs, wherein each primer pair is complementary to a different polymorphic region of the nucleic acid of SEQ ID NO:1.

9. A method of diagnosing predisposition to low spine bone mineral density, low total hip bone mineral density, low femoral neck bone mineral density, or osteoporosis in a human, said method comprising the steps of:
obtaining a nucleic acid sample from the human; and
detecting the presence or absence of at least one allelic variant of a polymorphic region in a nucleic acid having a sequence as set forth in SEQ ID NO:1 in the sample,
wherein the polymorphic region corresponds to the polymorphic site at position 245 of SEQ ID NO:1.

10. A method of diagnosing predisposition to low total hip bone mineral density, low femoral neck bone mineral density, or osteoporosis in a human, said method comprising the steps of:
obtaining a nucleic acid sample from the human; and
detecting the presence or absence of at least one allelic variant of a polymorphic region in a nucleic acid having a sequence as set forth in SEQ ID NO:1 in the sample,
wherein the polymorphic region corresponds to the polymorphic site at position 1470 of SEQ D NO:1.


SEQUENCE LISTING (21 sequences)

SEQ ID NO: 1 - DNA, 8778 bp, Homo sapiens
ctgctgctattgctcctccggccgcggccgctgccgtcgcttcggcacccgccgccctcacctcccttacccctcccggtgccgccgcaaaaccagtcccgcggccgccaagcgatccctgctccgcgcgacactgcgtgcccgcgcacgcagagaggcggtgacgcactttacggcggcagcgtaagtgcgtgacgctcgtcagtggcttcagttcacacgtggcgccagcggaggcaggttgmtgtgtttgtgcttccttctacagccaatatgaaaaggcctagtaagtggggtcgggaggcgggcgtggagggacccacgtctggaagttgctgcagccaccacgacgctcttctacggctacggctttgtctctgctggtatgggggtgggagcctacgcgtaggccttggccctatttcctggtagaaccgagagttggaagtccctacggcgatcatgttaaccgcgcgggctcattctgcggaacgaagccgggcagagggtggggaagactaggctagattttcgtaaggaagcagcgtctgagccaggtttgaggcccaatattttctttccgtggccacgtgcagactggcccaggtgagagctgagaatcgcctcccagactcagtgttcctctcctgccttatgattcgtgctgtttgacacgaagtggttgtcgttttgtgtctcatacgctgttgtgtatgatcccattctaatattgtgagggtaagtgcagggaattttgactccattctggatctactgaatttaattctctgggatttgaaagtagcacgtatgtttgcattaggcatttcgcattagacttaacgttaggtttggtagccaataacacaagaaaaggatataactccatagtgcgttaacccagaactaatcatttgggttaacagatttgtgatgtgtttctttgtagagttaaagaaagcaagtaaacgcatgacctgccataagcggtataaaatccaaaaaaaggtaagtgtagtgcttgagagagctgtaccaaacacattgctaaactgattttgccctgttcctttgcgggaaagtctgggttaatgtgatttggttttgggaaatggcattggatagactgaccatgggcacaagctcttaggcatcaggagtgcagctgtgagaaagtgcagtgatttggtgataagtctctaaatttgttcagcatgttaatctctgcatagagagccttctagttacaatttcttgctgttttatacttacatatgcattactttgtaagattccaattaaagctccattttcctaggacattttataggcataactaaattgcagccagattggtttctcacttgaattctgcttaagtataaagatatttttgtaagcagacaaaatctctttattttaataggttcgagaacatcatcgaaaattaagaaaggaggctaaaaagcrgggtcacaagaagcctaggaaagacccaggagttccaaacagtgctccctttaaggaggctcttcttagggaagctgagctaaggaaacagagggtaagttatgttagccagaattttcattgagtggtgtagtgtgttatgtgtgatatttttcagagtaaggtaacaacactagtcactggttcacctatttcccttatggctctgacagcttgaagaactaaaacagcagcagaaacttgacaggcagaaggaactagaaaagaaaagaaaacttgaaactaatcctgatattaagccatcaaatgtggaacctatggaaaaggtatgattaggtctctttatgaatgagagatcaggggttttgattttggtttttttgcttgggcctgagtgcagtggcacaatcacaactcactgcaacctggaccacccaggctcaagcagtcttaccacctcagtctccaagtagctgggactacagaagcacaccatcacgcctggctaattttttttagcagacacgggctttcactatattgccaaggctggtctcaagtgatccacccaactcagcctcccgaagtttctggtattacaggtgtgggctgttgtgcctggctgagagatgagttctgatgcagaaataaaagcacatccacaggctgctgagcttcttgggaggaagacaactgagttcagactccatcttacctatttaacaactgcaagggctgctactcagctgtggaaaatggagttagaggttacagttgctctacttctaattttgtgttatttccccctttatcctctaggagtttgggctttgcaaaactgagaacaaagccaagtcgggcaaacagaattcaaagaagctgtactgccaagaacttaaaaaggtatcttagcctaggtcagtgtctgacagtagtaatgaggtttaaaagactcaagtcattttttttttaaccttttaagatgaagggtgcatgtgcaggtttgttatatgggtaaacttgcgtcataggggtttgctgtacagattatttcatcacccaggtattaagcctagtacgacattagttatctttcctgatcctctccctcctcccaccttttcaccaacaaatcatttcgtgactcgactctagcttatgctgtttaatgcctttcctgctatgtttacctgacggaaatagtttctttggttctaaatatttgcacaaaactggttctgcctgtaagcatgatttacaacattaaaaaaaaacgttgacatagtgttgagattgagaaaggtacattggagtaagcagtgtcaggctaaaggtctctaaagtactctgttgaaacctaagtgaaggaggacaacttggtgtagttgctctccagcactcccatccccaacatccattttcccaagctcactcccccatggatagaacctgactgcccctcagcagcttttggcaaggccagaaggacctatcaaactatataattcactatgggaggattcagacagggatatttgcatttttgaaatccatcttgatcagagactgctgagcaagcctatgttttactttcctgtgtgagaaatgatgagggtcaacattcttcataccaaagtgaagacatgagatccaactctgagctcaccctgttgctaaatggataatgccagtactctcttgtggaaggtattaccagaacaagggatgtagttctgatcattttctccttgataatgtagttctggtcattttttccttgataggtgattgaagcctccgatgttgtcctagaggtgttggatgccagagatcctcttggttgcagatgtcctcaggtagaagaggccattgtccagagtggacagaaaaagctggtacttatattaaataaatcaggtgagtaaagagggtaccctttgtcttctgtgtacatgggtgaggtacgaggaaacagtctgatagtcactgaagactgattagatccaactctgatctcagcaaagccagagtacgtgcactttgccagagacagtgctaggcagtggggagccaggtgacttttacaactgactcaactggtttctactattcttttgccattcagtatttaccatcttttaaataaagagtgtaagctgctatacccagcttattgtgtagtatatttcatctaggaagtgatgacagtgtgacaaattccccacacctacacaatgtcgggtattagttcaagagtgaaataaattggaacgtatgtgacaaaatatttaaatgaaatgcataattatgcatctgagtttgagcagcaggaaaaagaaaacccagaacagagaattacaaagcagaaaatgggaatgagatctaaaattgttgttggggttaagaaacaattggctgcttgggaggctgaagtgggcacatcacttgaggccaggagttcgagaaaagcctggccaacacggcgaaaccccatctctactaaaatacaaatattagccgggcatgatgatgggcacttgtagtcccagctactcggaaggctgaggcaggagaattgcttgaacccgggaggcggaggttgcagtgagccgatactgtgccactgcactccagcctgggcaacaacacttcgtctcaaaaatgaagaaacaattggctgctagctaaaggtaaattctggaaacatagctctagggttagtagggttgtaatccagacgttgagtctgtcctgaagttttcaagtgagcaatacaaggggaattgaaatagagaagtgcagatgctgagctctgttaagatacgggcagtatggtaggggagcttaccctgccctgattttctagttaaatccttttgaaaggactgggaaaatgtaaaccagagtaaaatctatagttgccagattttgcaaatgcatctcaacaaaatagccacattggagcaaatgtctttttctttttttctttttgagatggaggcttgctgtgtcacccaggctggagtgcagtggcgccatctcagctcactgcaagctccacctcccgggttcacgccgttctcctgcctcagcctcccgagtagctgggactacaggtgcccaccaccacgcccagctaatttttttgtatttttagtagagacggggttttatcgtgttagccaggatggtcttgatctcctgaactcgtgatccgcctgtctcggcctcccaaagtgctgggattacaggcgtgagccaccgtgcctggccgcaaatgtcttatttctaattgctatcagatctggtaccaaaggagaatttggagagctggctaaattatttgaagaaagaattgccaacagtggtgttcagagcctcaacaaaaccaaaggataaagggaagataaccaaggtatcctttattagtggtaagaaatgtgattctttcagattttggttgaaatatgatgagtgtacaaaatcttgatttaagtgaatgaaaaattacaagatccaactctgatttcagccagagatcatctgaaaggcaatgtagttatcttaagagctgggctctggagcctgattgcttggggtttgttgaaatttatcaggtaagttgccagaataattcaactatttggaattttagcgtgtgaaggcaaagaagaatgctgctccattcagaagtgaagtctgctttgggaaagagggcctttggaaacttcttggaggttttcaggaaacttgcagcaaagccattcgggttggagtaattggtgagtttcagttcattactttttactttttaagtgttgaaatagttaagaagtttagccagctctccaagtgcccaagcagcagtgtatggagttgttgtcaagtaaaaggctcactcaaatactagcttcttgcttataccttactgaaagcacataaccacacacaatttaaagaaaaaaacttacacaactgctgccagattcaggttttttgttgggactgattactgtaggaagctggtttctaaaagttcttggtttgtttgaatttatagtattttcctgcctttgcattacttgtgcaagaaatgaagaaactaaaattggtcttagtattgaagtgaagacactgagatccaactctgatcttgccctaaacatcagggaaatggaaaattaggcagtgaaaatttcataagtgccaacatataatgctttttatatctggatttcccatttatttgtaggtttcccaaatgtggggaaaagcagcattatcaatagcttaaaacaagaacagatgtgtaatgttggtgtatccatggggcttacaaggtaaatggaggtgtccataattgtaatattatagtgacacactattttattttggttatctcaaggaaggtgattttttttttttttttttttgagacagtttcactcttgttcccaggctggagggcaatggcgcaatctcggctcactgcaacctccacctctcaggttcaagtgattctcctgcctcagcctccagagtagctgggattacaggcatgtgcccccacgcccggctaattttgtatttttaatagagacgggtttctccatgttggttaggctggtctcaaactcctgacttcagctgatctgcccgcctcggcctcccaaagtgctggggttacaggccaagccaccgcgcccggcctatttttattttttaaagcttttggtgaaagcagagatttaagccagtgctagaactgattggagtgggacagctgcccacaatttagtttgaaagacaagttcagcagatagattgagagagaggaaagctccctcagaggaagtgatgtttgaacctggccttgagaaattaatagaaatttgccagatagaagtctgccaccacacctggctagtttttgtatttttaatagagatggggtttcactgttggccaggctagcctcaaactcctggccttaagtcatccacccgcctaggcctcacaaagtgttgggattacacttgtgttgggtggcatgagccactgtgcctggccaacttttagattttttttttttgggagatggagtctgtctcccaggctggagtacagtggtgctatcttggctcactgcaacctcagcctcctgagtagctgggatcaaaggcacccggctaatttttgtatttttagtagagacagggctttcatcatgttggccaggctggtctcaaactcctgacctcaggtgatccacccacctcggcctcccaaagtgctgggattacaggcgtgagccaccgcgcccagccagaaggtggcatatttatagcaaaggaaatagcatgtgttttggttgatgtaaatatataagctataacatgtagtgttctctttagaacagtcgggtatgctgttacagttttagataaatgtgaagcaaatgatgataaactggatctgactgactgtgctgagtctgttcaatccaaccctgagcttcatgttctgtctcttaacctccaaatagaccaatgcccccatcaattccatcgctcttctttcaggagcatgcaagttgtccccttggacaaacagatcacaatcatagatagtccgagcttcatcgtatctccacttaattcctcctctgcgcttgctctgcgaagtccagcaagtattgaagtagtaaaaccgatggaggctgccagtgccatcctttcccaggctgatgctcgacaggtaaaaggaccccttctcatgagctccttggagccatcttctttcatcataagcattttgagtagaaaaatcttggaagtgttttaaagtactggcatgtcagatgaaggacagctcctttgtttggttttttttttaaggtagtactgaaatatactgtcccaggctacaggaattctctggaattttttactgtgcttgctcagagaagaggtatgcaccaaaaaggtggaatcccaaatgttgaaggtgctgccaaactgctgtggtctgagtggacagggtaagctttcttttctgttggcattttggtgaccactagaataaaccttcttttgacacatcttatttttaatatcagtgcctcattagcttactattgccatccccctacatcttggactcctcctccatattttaatgagagtattgtggtagacatgaaaagcggcttcaatctggaagaactggaaaagaacaatgcacagagcataagaggtgagaattgtgtgtcgctgctgtcttcatcagctgacaggccagtggagctcttacctgtttacatgggcttgctttctttcccagccatcaagggccctcatttggccaatascatccttttccagtcttccggtctgacaaatggaataatagaagaaaaggacatacatgaagaattgccaaaacggaaagaaaggaagcaggaggagagggaggatgacaaagacagtgaccaggaaactgttgatgaagaagttgatgtaagtgtgtcctccatgagttaaaactgaagtgagttttctagcattataatacataatggaaggaactgaagataggaaatatttgaggcttgtgatccattagccttaattttgcacatcccgttatatgtacctccaaagagttaatttttcaggtacataactacttggattaaatgagcagacaagggctactaatccagcactatttttctttgtcacacaggaaaacagctcaggcatgtttgctgcagaagagacaggggaggcactgtctgaggagactacagcaggtgaggcaggcaaaaggggttctaacgaagcagcatggtatagaatcacttttactttttgaaaatctctttattttcctgcaatataggtgaacagtctacaaggtcttttatcttggataaaatcattgaagaggatgatgcttatgacttcagtacagattatgtgtaacagaacaatggctttttatgattttttttttaacattttaagcagactgctaaactgttctctgtataagttatggtatgcatgagctgtgtaaattttgtgaatatgtattatattaaaaccaggcaacttggaatccctaaattctgtaaaaagacaattcatctcattgtgagtggaagtagttatctggaataaaaaaagaagatacctattgaaaaatgtaagttttatttacagatcaggccacaggttacaaaattaaaaccaacagcagttttgaattatctgtaccagctagctgaactagccatatcagttc

SEQ ID NO: 2 - DNA, 15 bp, Homo sapiens
agcggaggcaggttg

SEQ ID NO: 3 - DNA, 15 bp, Homo sapiens
ggaagcacaaacaca

SEQ ID NO: 4 - DNA, 13 bp, Homo sapiens
aggttgatgtgtt

SEQ ID NO: 5 - DNA, 13 bp, Homo sapiens
aggttgctgtgtt

SEQ ID NO: 6 - DNA, 13 bp, Homo sapiens
aacacatcaacct

SEQ ID NO: 7 - DNA, 13 bp, Homo sapiens
aacacagcaacct

SEQ ID NO: 8 - DNA, 15 bp, Homo sapiens
tgtgtttgtgcttcc

SEQ ID NO: 9 - DNA, 15 bp, Homo sapiens
caacctgcctccgct

SEQ ID NO: 10 - DNA, 15 bp, Homo sapiens
aggaggctaaaaagc

SEQ ID NO: 11 - DNA, 15 bp, Homo sapiens
ggcttcttgtgaccc

SEQ ID NO: 12 - DNA, 13 bp, Homo sapiens
aaaagcagggtca

SEQ ID NO: 13 - DNA, 13 bp, Homo sapiens
aaaagcggggtca

SEQ ID NO: 14 - DNA, 13 bp, Homo sapiens
tgaccctgctttt

SEQ ID NO: 15 - DNA, 13 bp, Homo sapiens
tgaccccgctttt

SEQ ID NO: 16 - DNA, 15 bp, Homo sapiens
gggtcacaagaagcc

SEQ ID NO: 17 - DNA, 15 bp, Homo sapiens
gctttttagcctcct

SEQ ID NO: 18 - DNA, 20 bp, Homo sapiens
ctcgtcagtggcttcagttc

SEQ ID NO: 19 - DNA, 22 bp, Homo sapiens
ccttttcatattggctgtagaa

SEQ ID NO: 20 - DNA, 22 bp, Homo sapiens
aataggttcgagaacatcatcg

SEQ ID NO: 21 - DNA, 20 bp, Homo sapiens
ttggaactcctgggtctttc